(19) United States
(12) Patent Application Publication
(10) Pub. No.: US 2024/0076593 A1
(43) Pub. Date: Mar. 7, 2024

(54) METHODS FOR ORGANOID PASSAGING USING MICROPLATE WELL UNITS

(71) Applicant: Molecular Devices (Austria) GmbH, Puch bei Hallein (AT)

(72) Inventors: Josef ATZLER, Hallein (AT); Felix SPIRA, Strasswalchen (AT)

(73) Assignee: Molecular Devices (Austria) GmbH, Puch bei Hallein (AT)

(21) Appl. No.: 18/262,330

(22) PCT Filed: Feb. 18, 2022
(86) PCT No.: PCT/IB2022/051484

§ 371 Date: Jul. 20, 2023

Related U.S. Application Data
(60) Provisional application No. 63/151,082, filed on Feb. 19, 2021.

(51) Int. Cl. C12M 3/00; C12M 1/00; C12M 1/32; C12N 5/071

(52) U.S. Cl. CPC C12M 21/08; C12M 23/12; C12M 47/02; C12M 47/12; C12N 5/0602; C12N 2513/00

(57) ABSTRACT

Disclosed are various embodiments related to automated cellular passaging of spheroids, tumoroids, organoids and/or other multi-cellular bodies. Organoids can be cultured in a hydrogel that is disposed in a well unit of a microwell plate. The well unit includes a primary well section that is fluidly connected to a secondary well section via at least one channel, and the hydrogel is disposed in the primary well section of the well unit. The hydrogel is dissipated into hydrogel fragments, thereby separating the organoids from the hydrogel. The hydrogel fragments are removed from the well unit, while the organoids remain in the well unit. The remaining organoids are broken into organoid fragments and corresponding debris. A fresh culture environment is created using the organoid fragments.

### CROSS REFERENCE TO RELATED APPLICATIONS

This application is being filed on Feb. 18, 2022, as a PCT International Patent Application that claims the priority of and benefit to U.S. Provisional Application No. 63/151,082, filed on Feb. 19, 2021, the entire disclosure of which is incorporated by reference in its entirety.

### BACKGROUND

Culturing cells in a three-dimensional (3D) environment yields cellular behavior and morphology that more closely matches what is observed in the human body. 3D hydrogels/hydroscaffolds used for this kind of culturing have a unique attribute: cells can be deposited in specific locations in 3D space and remain in position for extended time periods. This enables the creation of structures (e.g., embryoid bodies, fused embryoid bodies, spheroids, tumoroids, organoids, and/or other multi-cellular bodies) and co-culture environments where cellular interactions and developments over time are observed.

### SUMMARY

Aspects of the present disclosure are related to automated passaging of the spheroids, tumoroids, organoids and/or other multi-cellular bodies. According to various embodiments, as organoids and other multi-cellular bodies grow and develop over time in the wells of a microplate, passaging is required to remove debris associated with dead cells and toxic-by products, reduce total cell numbers, obtain a homogenous population of smaller organoids, and facilitate continued cell growth and propagation. The passaging method of the present disclosure improves on traditional methods associated with organoid or multi-cellular body passaging by reducing time, reducing costs, and eliminating the need for additional laboratory equipment (e.g., conical tubes, centrifuge, etc.) and centrifugation.

In one embodiment, among others, a method for passaging organoids includes culturing one or more organoids in a hydrogel that is disposed in a well unit of a microwell plate. The well unit includes a primary well section that is fluidly connected to a secondary well section via at least one channel, and the first hydrogel is disposed in the primary well section of the well unit. The hydrogel is dissipated into hydrogel fragments, thereby separating the one or more organoids from the hydrogel. The hydrogel fragments are removed from the well unit while the one or more organoids remain in the well unit. The one or more organoids are broken into the organoid fragments and corresponding debris. A fresh culture environment including the organoid fragments is created.

In one or more aspects, the at least one channel is formed by at least one gap between a bottom surface of the well unit and a bottom portion of a shared sidewall of the primary well section and the secondary well section. In some examples, the height of the at least one gap can be sized between about ten (10) microns up to about one hundred (100) microns. In various aspects, dissipating the first hydrogel into hydrogel fragments further comprises cooling the hydrogel. The cooling of the hydrogel can further comprise dispensing a liquid medium into the well unit. In some aspects, the liquid medium is at a temperature of about 10 degrees Celsius or less. In some aspects, the temperature is about 4 degrees Celsius or less.

In various aspects, the method further includes collecting the one or more organoids from the primary well section of the well unit via a liquid handler and applying at least one shear force to the one or more organoids within the liquid handler. The one or more organoids can be broken into organoid fragments and corresponding debris as a result of the at least one shear force. In various aspects, the method can further include depositing the organoid fragments and corresponding debris into the primary well section of the well unit via the liquid handler. In one or more aspects, applying the at least one shear force comprises manipulating the liquid handler to cause the one or more organoids to move vertically within the liquid handler.

In various aspects, removing the hydrogel fragments can include inserting a liquid handler into the secondary well section of the well unit, transferring the hydrogel fragments from the primary well section into the secondary well section of the well unit via the at least one channel, and collecting the hydrogel fragments from the secondary well section via the liquid handler. In one or more aspects, the method includes flushing the at least one channel to remove at least one of a debris fragment or an organoid fragment from the at least one channel. In one more aspects, the method includes moving the corresponding debris to the secondary well section of the well unit via the at least one channel.

In various aspects, creating the fresh culture environment including the organoid fragments further includes collecting the organoid fragments via a liquid handler and depositing a fresh hydrogel on a bottom surface of the primary well section of one of another well unit or the well unit. In some aspects, the organoid fragments are embedded in the fresh hydrogel prior to depositing the fresh hydrogel on the bottom surface of the primary well section. In other aspects, the organoid fragments are embedded in the fresh hydrogel after the fresh hydrogel is deposited on the bottom surface of the primary well section. In one or more aspects, the microwell plate including the well unit is placed in an incubator.

In another embodiment, among others, a method includes separating one or more cellular bodies from a hydrogel that is disposed on a bottom surface of a first well unit of a first microplate plate. The first well unit includes a culture well and a supply well that are fluidly connected to one another via at least one channel, and the first hydrogel is disposed within the culture well of the first well unit. The hydrogel can be removed from the first well unit via a supply well of the well unit while the one or more cellular bodies remain within the culture well of the first well unit. The one or more cellular bodies can be broken into cellular body fragments and corresponding debris. The corresponding debris can be removed from the supply well of the first well unit, while the cellular body fragments remain within the culture well of the first well unit. A fresh culture environment in is created in one of the first well unit or a second well unit, and the fresh culture environment includes the cellular body fragments.

In various aspects, the at least one channel is sized and shaped to prevent passage of objects sized greater than about twenty-five microns between the culture well and the supply well. In various aspects, separating the one or more cellular bodies from the hydrogel further comprises cooling the hydrogel to a temperature that causes the hydrogel to liquify. In one or more aspects, the method includes removing the one or more cellular bodies from the culture well via a pipette and applying one or more shear forces to the one or more cellular bodies via the pipette. The one or more cellular bodies can be broken into cellular body fragments and corresponding debris based on the one or more applied shear forces. In various aspects, creating the fresh culture environment in one of the first well unit or the second well unit further includes depositing a fresh hydrogel in the one of the first well unit or the second well unit, where the cellular body fragments are embedded in the fresh hydrogel, and culturing the cellular body fragments being embedded in the fresh hydrogel.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views.

FIG. 1 illustrates an example of a cross section of a well unit of a microplate associated with the cell culturing according to various embodiments of the present disclosure.

FIGS. 2A-2B illustrate examples of cross-sectional views of the well unit of FIG. 1 while depicting a workflow for removing a liquid medium from the well units of FIG. 1 according to various embodiments of the present disclosure.

FIGS. 3A-3H illustrate examples of cross-sectional views of the well units of FIG. 1 where each figure represents an example of a workflow step associated with cellular passaging according to various embodiments of the present disclosure.

FIG. 4 illustrates a flowchart of an example method related to the cellular passaging in accordance to various embodiments of the present disclosure.

### DETAILED DESCRIPTION

The present disclosure relates to a method for automated passaging of the spheroids, tumoroids, organoids and/or other multi-cellular bodies. Microwell microplates are used for the growing, culturing, monitoring, and analyzing of embryoid bodies, fused embryoid bodies, spheroids, organoids, and/or other multi-cellular bodies in vitro. According to various embodiments, as spheroids, tumoroids, organoids and other multi-cellular bodies grow and develop over time in the wells of a microplate, passaging is required to remove debris associated with dead cells and toxic-by products, reduce total cell numbers, obtain a homogenous population of smaller organoids, and facilitate continued cell growth and propagation. The passaging method of the present disclosure improves on traditional methods associated with spheroid, tumoroid, organoid or other multi-cellular body passaging by reducing time, reducing costs, and eliminating the need for additional laboratory equipment (e.g., conical tubes, centrifuge, etc.) and centrifugation. In addition, according to various embodiments, the passaging method of the present disclosure improves on traditional methods by being able to control and adjust the size range of the organoids or multi-cellular bodies thereby providing a more controlled and homogenous population.

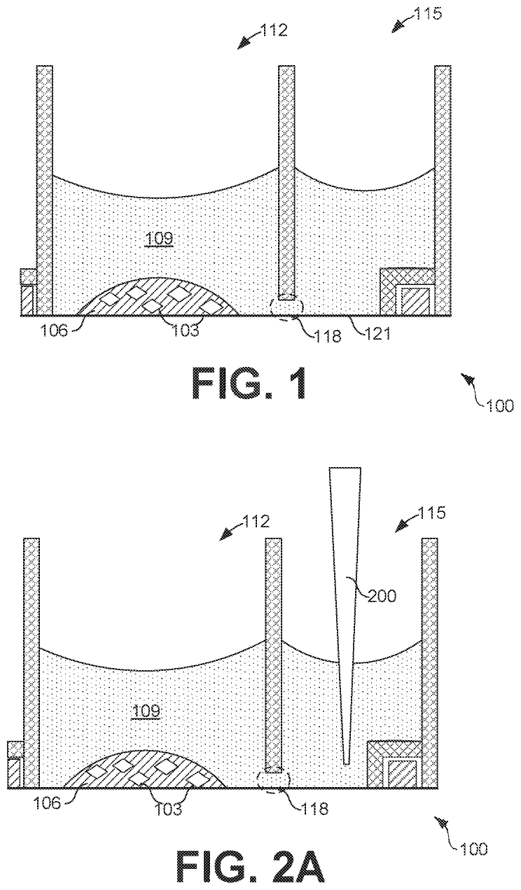

According to various embodiments, the passaging method of the present disclosure uses microplates having well units comprising two conjugated wells that are fluidly connected to one another via the at least one channel, including the microplates which are described in U.S. Provisional Application 63/094,946 entitled “Microplates for Automating Organoid Cultivation” filed on Oct. 22, 2020, and U.S. Provisional Application 63/131,123 entitled “MICROPLATE WELLS FOR CELL CULTIVATION” filed on Dec. 28, 2020, both of which are incorporated by reference herein in their entirety. FIG. 1 illustrates an example of a well unit 100 of a microplate that can be used in connection with the passaging method of the present disclosure. In particular, FIG. 1 illustrates an example view of a well unit 100 of a microplate that can be used for growing, culturing, monitoring, and assaying embryoid bodies, fused embryoid bodies, spheroids, organoids, or other multi-cellular bodies in accordance to various embodiments of the present disclosure.

As shown in FIG. 1, cellular bodies 103 (e.g., spheroids, tumoroids, organoids, etc.) are embedded in a dome of hydrogel 106 (e.g., Matrigel®) that is disposed in the well unit 100 and is surrounded by a liquid medium 109. According to various embodiments, the liquid medium 109 can contain the appropriate growth factors and supplements to create and/or stimulate growth of the desired multi-cellular body. Exemplary growth factors that can be suitable include angiopoietin, bone morphogenetic proteins (BMPs), ciliary neurotropic factor, colony stimulating factors, ephrins, epidermal growth factor, erythropoietin, fibroblast growth factors, glial-derived neurotrophic factor, hepatocyte growth factor, insulin, insulin-like growth factors, interleukins, leukemia inhibitory factor, keratinocyte growth factor, neuregulins, neurotrophins, platelet-derived growth factor, transforming growth factors, tumor necrosis factor (alpha), vascular endothelial growth factor, and/or the like.

According to various embodiments, the well unit 100 of FIG. 1 comprises a primary well section 112 and a secondary well section 115. In various examples, the primary well section 112 and the secondary well section 115 are in fluid connection with one another via at least one channel 118 to facilitate a gravitational flow of liquid (e.g., liquid medium 109) between the primary well section 112 and the secondary well section 115 in response to a tilting of the microplate. Exchanging the liquid medium 109 between the primary well section 112 and the secondary well section 115 removes toxic by-products and supplies the growing cell cultures with fresh nutrients. In the example of FIG. 1, the hydrogel 106 and the cellular bodies 103 are disposed in the primary well section 112 of the well unit 100.

According to various examples, the at least one channel 118 is further sized and shaped to prevent objects having dimensions (e.g., diameter, height, width, etc) of a certain size (e.g., greater than about twenty-five (25) microns (μ)) from passing from one well section to the other well section. In some examples, the height of the at least one channel 118 can be sized between about ten (10) microns up to about one hundred (100) microns. In other examples, the height of the at least one channel can be sized between about ten (10) microns to about twenty-five (25) microns. For example, where the height of the at least one channel 118 is about 25 microns, objects (e.g., spheroids, tumoroids, organoids, organoid fragments, etc) that are sized at about 25μ or greater that are present in the primary well section 112 will be prevented from migrating to secondary well section 115. However, corresponding debris (e.g., toxic by-products, dead cells, other corresponding debris that are a result of culturing the cellular bodies 103, cellular body fragments that are to be removed as a result of reducing total cell numbers and to obtaining a homogenous population of smaller organoids, etc) that are present in the primary well section 112 and have dimensions (e.g., diameter, height, width, etc) that are less than or equal to the certain size (e.g., about 25μ) are able to pass through the at least one channel 118 and into the secondary well section 115. As a result, the corresponding debris can be aspirated or otherwise collected using a liquid handler (e.g., pipette) via the secondary well section 115 without disturbing the cellular bodies 103 within the primary well section 112.

According to various embodiments, the primary well section 112 is sized and shaped to support deposited cellular bodies 103 (e.g., cell aggregates) that can be embedded in a hydrogel 106 that is deposited into the primary well section 112. For example, the primary well section 112 can be considered a culture well that is used to grow the embryoid bodies, fused embryoid bodies, spheroids, organoids, and/or other multi-cellular bodies, as can be appreciated. According to various embodiments and dependent upon a number of well units 100 in the microplate, the width of the primary well section 112 can be up to about 8 millimeters (mm) (e.g., for 96 well plate), up to 11 mm (e.g., for a 48 well plate), up to about 17 mm (e.g., for a 24 well plate), and/or other sizes as can be appreciated. In addition, the depth of the primary well section 112 and the secondary well section 115 is specified such that the microplate can be tilted to allow fluid exchange within the well units 100 without spilling the fluid out of the respective primary well section 112 or secondary well section 115 of each the well units 100.

The secondary well section 115 can be used to supply feeding media and/or other nutrients that can be used to feed the growing cell aggregates positioned in the primary well section 112. In addition, the secondary well section 115 can be used to harvest supernatant from the cell aggregates, as can be appreciated. For example, the secondary well section 115 can be considered a supply well that comprises the feeding media and/or other nutrients that can be used by the growing cell culture in the primary well section 112. The secondary well section 115 is sized and shaped to hold fluid that can be exchanged with the primary well section 112 according to various embodiments of the present disclosure. According to various embodiments and dependent upon a number of well units 100 in the microplate, the width of the secondary well section 115 can be up to about 8 millimeters (mm) (e.g., for 96 well plate), up to 11 mm (e.g., for a 48 well plate), up to about 17 mm (e.g., fora 24 well plate), and/or other sizes as can be appreciated.

According to various embodiments, the size and shape of the primary well section 112 and the secondary well section 115 can differ from one another. In some examples, the primary well section 112 is larger (in a dimension, for example diameter or volume) than the secondary well section 115. In other examples, the secondary well section 115 is larger than the primary well section 112. In some examples, the primary well section 112 comprises a shape that differs from a shape of the secondary well section 115.

According to various embodiments, the well unit 100 further comprises a bottom layer sheet 121 disposed on an underside of a well plate body of the microplate. The bottom layer sheet 121 is attached to the underside of the well plate body forming the bottom surface of the well unit 100. In various examples, the bottom layer sheet 121 comprises a viewing window that is optically transparent to allow for imaging of spheroids, organoids, or other cell cultures being cultured in the well unit 100 of the microplate, as can be appreciated. The viewing window can be a window that is suitable for microscopic observation, whether brightfield, phase-contrast, fluorescent, confocal, two-photon, or other microscopic imaging modalities as known in the art.

In various examples, the bottom layer sheet 121 comprises a gas permeable sheet that is configured to increase an oxygen supply for the growing spheroids, organoids, or other cellular bodies in the well unit 100 of the microplate. The gas permeable sheet can be formed of a material comprising polytetrafluoroethylene (PTFE), PEFP, Polyimide, Polydimethylsiloxane (PDMS), Polycarbonate, and/or other material as can be appreciated. According to various examples, the gas permeable sheet can have a thickness of about 5-70 microns. According to various examples, the gas permeable sheet can comprise a plurality of pores. In other examples, the gas permeable sheet can allow molecules to pass by diffusion. Alternatively, the gas permeable sheet can comprise some other thickness, pore diameter, and pore density.

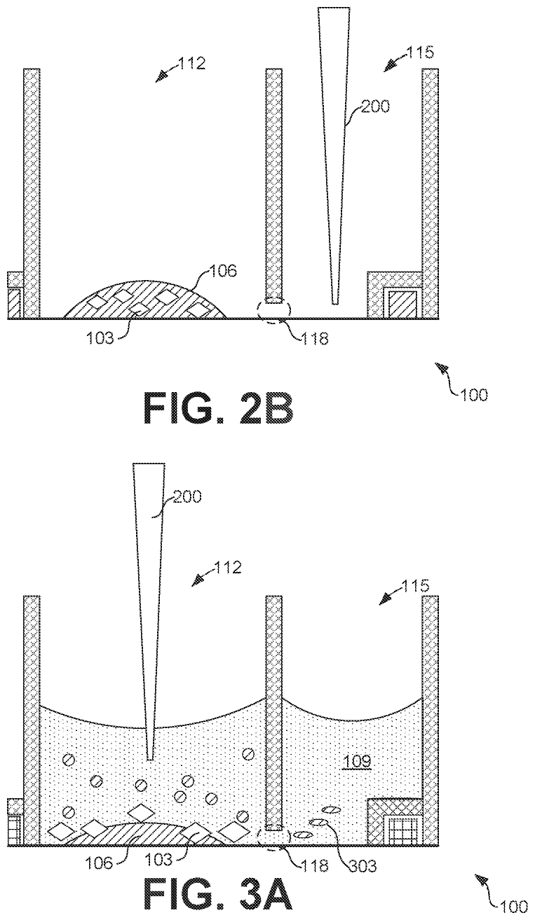

Turning now to FIGS. 2A and 2B, shown are examples of a workflow associated with removing a liquid medium 109 from the well unit 100 in accordance of various embodiments of the present disclosure. In particular, similarly to FIG. 1, FIG. 2A illustrates a culturing environment including cellular bodies 103 being embedded within a hydrogel 106 that is disposed on a bottom surface (e.g., the bottom layer sheet 121) of the primary well section 112 of the well unit 102. A liquid medium 109 is also illustrated in both the primary well section 112 and the secondary well section 115 of the well unit 100. As can be appreciated, the liquid medium 109 can comprise the appropriate growth factors and supplements to create the desired cellular bodies 103.

The at least one channel 118 of the well unit 100 provides a fluid connection between the primary well section 112 and the adjacent secondary well section 115. The at least one channel 118 further provides the ability to facilitate a continual gravitational flow of the liquid medium 109 via the tilting of the corresponding microplate and allow for advance feeding of the cellular bodies 103 (e.g., organoids or other multi-cellular bodies). In various examples, the liquid medium 109 (e.g., feeding media and/or other nutrients) can be introduced into the secondary well section 115 and ultimately introduced into the primary well section 112 via the at least one channel 118. In various embodiments, the liquid medium 109 can be added and/or removed from one of the well sections (e.g., secondary well section 115) using a liquid handler 200 (e.g., pipette) without disturbing the environment in the well (e.g., primary well section 112) of interest.

FIG. 2B illustrates an example of the well unit 100 following the removal of the liquid medium 109 using the liquid handler 200. As can be appreciated, the liquid medium 109 can be removed without disturbing the environment (e.g., cellular bodies 103 embedded in hydrogel 106) of the primary well section 112.

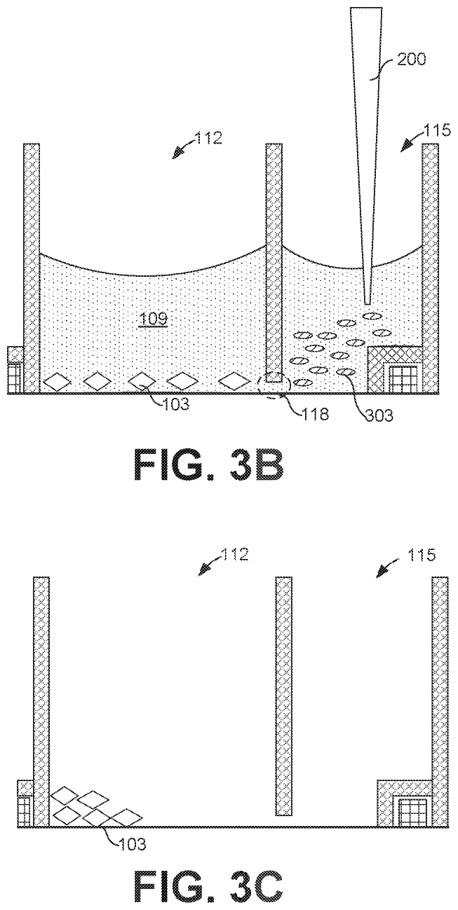

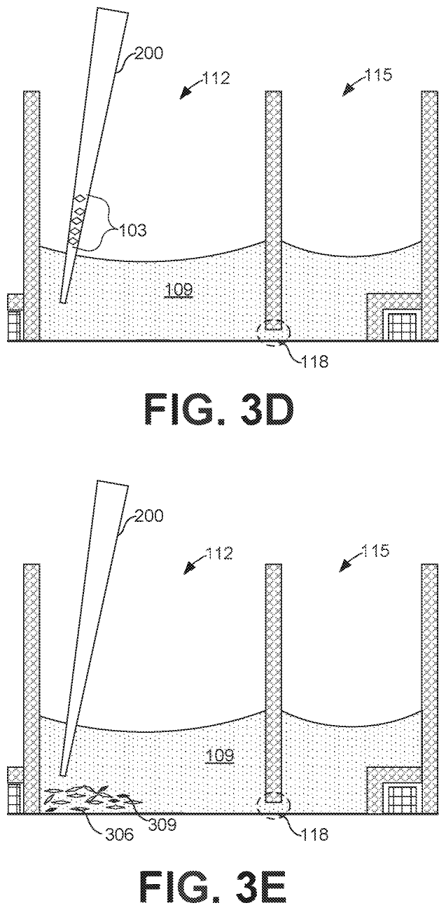

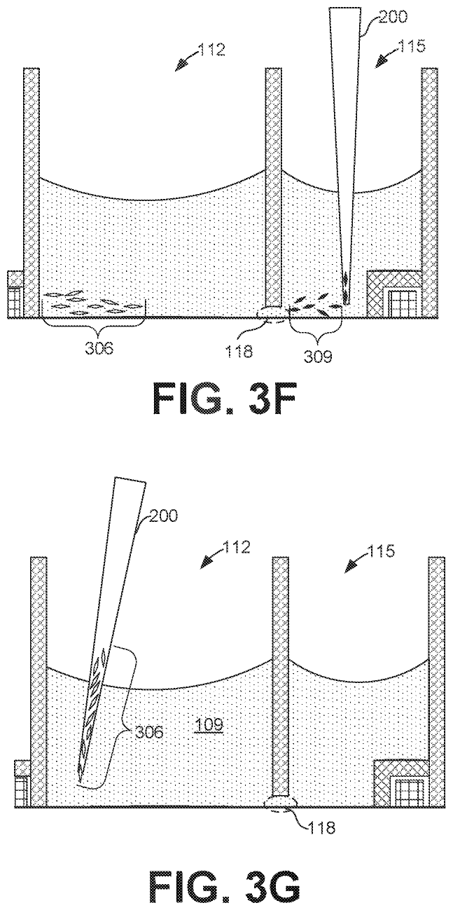

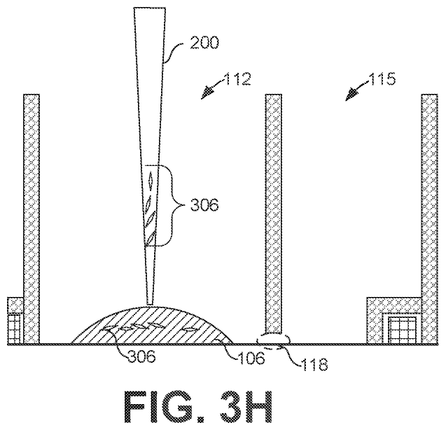

Next, a general description of the passaging workflow of the present disclosure is provided with reference to FIGS. 3A-3H. In particular, FIGS. 3A-3H illustrate cross-sectional views of the well unit 100 of a microplate. Each view corresponds to a respective step associated with the passaging workflow in accordance to various embodiments. According to various embodiments, as cellular bodies 103 grow over time in the well units 100 of a microplate, passaging is required to remove debris associated with dead cells and toxic by-products, reduce total cell numbers, obtain a homogenous population of smaller organoids, facilitate continued cell growth, and maintain the overall health of the cellular bodies 103.

As illustrated in FIG. 3A, the dome of hydrogel 106 can be broken down into a plurality of hydrogel fragments 303 or otherwise liquid form. For example, the solid hydrogel 106 can be transformed into a liquid, thereby separating the cellular bodies 103 from the hydrogel 106. In some examples, the solid hydrogel 106 is broken down into a plurality of hydrogel fragments 303 or otherwise liquid form by adjusting a temperature of the hydrogel 106 based on the melting properties of the hydrogel 106. In some examples, a hydrogel 106 can break down into a liquid format temperatures that are less than about 10 degrees Celsius (C). In this example, a liquid medium 109 having a temperature that is less than about 10 degrees (C.) (or less than about 4 degrees C.) can be introduced into the well unit 100 via the primary well section 112 or the secondary well section 115 using a liquid handler 200, as shown in FIG. 3A. In some examples, a microplate can be positioned on a temperature-controlled device (e.g., cooling plate) that is designed to adjust the temperature of the microplate such that the hydrogel 106 deposited within the corresponding well units 100 begins to liquify.

Turning next to FIG. 3B, the hydrogel fragments 303 or otherwise liquid form of the hydrogel 106 can be removed from the well unit 100 along with the liquid medium 109 via a liquid handler 200. As shown in FIGS. 3A and 3B, the plurality of hydrogel fragments 303 or otherwise liquid form of the broken-down hydrogel 106 can pass through the channel 118 formed between the primary well section 112 and the secondary well section 115. In addition, the dimensions of the at least one channel 118 prevent the passage of the cellular bodies 103 that are sized and shaped with dimensions that are greater than the dimensions of the at least one channel 118. As such, the cellular bodies 103 will remain in the primary well section 112 while the liquified hydrogel 106 and hydrogel fragments 303 pass through the at least one channel 118 and into the secondary well section 115. In order to preserve the cellular bodies 103 that have separated from the hydrogel 106, the liquified hydrogel 106, hydrogel fragments 303, and/or liquid medium 109 can be removed from the secondary well section 115 of the well unit 100.

In some examples, the workflow steps associated with FIGS. 3A and 3B can be repeated to allow for the hydrogel 106 to break down completely and allow for the complete removal of the hydrogel 106 from the well unit 100. In other words, washing of the cellular body suspension with the introduction and removal of a liquid medium 109 can be repeated multiple times to remove the hydrogel 106 from the well unit 100.

Once the hydrogel 106 is removed, only the cellular bodies 103 that were originally embedded in the hydrogel 106 remain in the well unit 100. This is illustrated in FIG. 3C. In some examples, a low level of liquid medium 109 can still be present in the primary well section 112 of the well unit. This liquid medium 109 can be removed as necessary. For example, the microplate can be tilted to allow the excess medium 109 to flow to the secondary well section 115 of the given well unit 100 for removal. The liquid medium 109 can be removed using the liquid handler 200, as can be appreciated.

Turning now to FIG. 3D, shown is an example of a workflow step associated with breaking up the cellular bodies 103 using harsh pipetting. In various examples, a liquid medium 109 can be introduced into the well unit 100 containing the cellular bodies 103. A liquid handler 200 can be used to aspirate or otherwise collect the cellular bodies 103 that remain in the primary well section 112 after the removal of the broken-down or otherwise liquified hydrogel 106. Once the cellular bodies 103 are aspirated into the liquid handler 200, shear forces can be applied to the cellular bodies 103, thereby causing the cellular bodies 103 to be broken down into a plurality of cellular body fragments 306 (e.g., spheroid fragments, tumoroid fragments, organoid fragments, etc) and corresponding debris 309. The cellular body fragments 306 comprises multi-cellular fragments of the cellular bodies 103 (e.g., spheroids, tumoroids, organoids, etc).

The shear forces that are applied can be a result of manual or automated manipulation of the liquid handler 200 to cause up and down movements of the cellular bodies 103 along a vertical axis of the liquid handler 200. The shear forces can cause the cellular bodies 103 to breakdown into a plurality of cellular body fragments 306 and corresponding debris. The shear forces can be applied to the cellular bodies 103 until a desired size of the cellular bodies 103 is obtained. In some examples, enzymes and/or chemicals (e.g., trypsin, a gentle disassociation medium, etc.) may be added to the well unit 100 or otherwise cellular bodies 103 prior to being aspirated by the liquid. In some examples, once the cellular bodies 103 are broken down into the multi-cellular body fragments 306 (e.g., organoid fragments), the cellular body fragments 306 can be placed in a well unit of a microplate and incubated in a cell dissociation reagent for a predetermined amount of time (e.g., up to about twenty minutes).

In various examples, a preferred size of the cellular body fragments 306 is around 25-500μ. However, it should be noted that other size fragments can be obtained. The corresponding debris 309 can comprise dead cells associated with the cellularly bodies 103 as well as toxic by-products that develop during the culturing of the cellular bodies 103.

Moving on to FIG. 3E, the plurality of cellular body fragments 306 and corresponding debris 309 can be reintroduced within primary well section 112 of the well unit 100 and liquid medium 109. In particular, FIG. 3E illustrates the plurality of cellular body fragments 306 and corresponding debris 309 that are a result of shear forces applied to the cellular bodies 103 that were originally embedded in the hydrogel 106. The corresponding debris 309 that is sized less than the dimensions of the at least one channel 118 will be able to pass through the at least one channel 118 and flow into the secondary well section 115. In various examples, tilting of the microplate can be required to cause the gravitational flow of the liquid medium 109 thereby allowing the corresponding debris 309 to enter the secondary well section 115.

Turning now to FIG. 3F, shown is an example of the removal of the corresponding debris 309 according to various embodiments of the present disclosure. In particular, the corresponding debris 309 having dimensions that are less than the dimensions of the at least one channel 118 are able to pass through the at least one channel 118 and enter into the secondary well section 115 where they can be removed via the liquid handler 200. In various examples, tilting of the microplate can cause the gravitational flow of the liquid medium 109 to move between the two wells allowing the liquid medium 109 and corresponding debris 309 to move through the channel 118 and into the different wells. In some examples, the addition of a liquid medium 109 and/or removal of the liquid medium 109 and debris 309 can be repeated as necessary to remove all debris 309 from the well unit 100.

Once the debris is removed from the well unit 100, only the cellular body fragments 306 remain in the well unit 100 along with, in some examples, some residual liquid medium 109. In some examples, additional liquid medium 109 can be provided within the well unit 100 and the cellular body fragments 306 can be aspirated into the liquid handler 200, as illustrated in FIG. 3G. The removal of the cellular body fragments 306 allow for the preparation of the fresh environment within the well unit 100.

In particular, once the cellular body fragments 306 are removed from the well unit 100, hydrogel 106 can be deposited in the primary well section 112 of the well unit 100. In some examples, the cellular body fragments 306 can be embedded in the hydrogel 106 prior to depositing the hydrogel 106 in a primary well section 112 of a well unit 100 of a microplate. In various embodiments, a new microplate will be used. In other examples, the cellular body fragments 306 are returned to the well unit 100 and deposited on the hydrogel 106 after the hydrogel 106 has been deposited in the well unit 100. FIG. 3H illustrates an example of depositing the cellular body fragments 306 onto the deposited hydrogel 106. In some examples, the microplate can be placed on a tray and heated to a desired temperature (e.g., about thirty-seven (37) degrees) and incubated for a desired amount of time (e.g., up to about twenty (20) minutes).

Once the hydrogel 106 and cellular body fragments 306 are introduced into the well unit 100, a liquid medium 109 can be added to the well unit 100 thereby providing a fresh environment to allow the cellular body fragments 306 to grow and develop as desired. The microplate can be placed in an incubator to stimulate the growth and development of the cellular body fragments. The passaging method can be repeated as necessary.

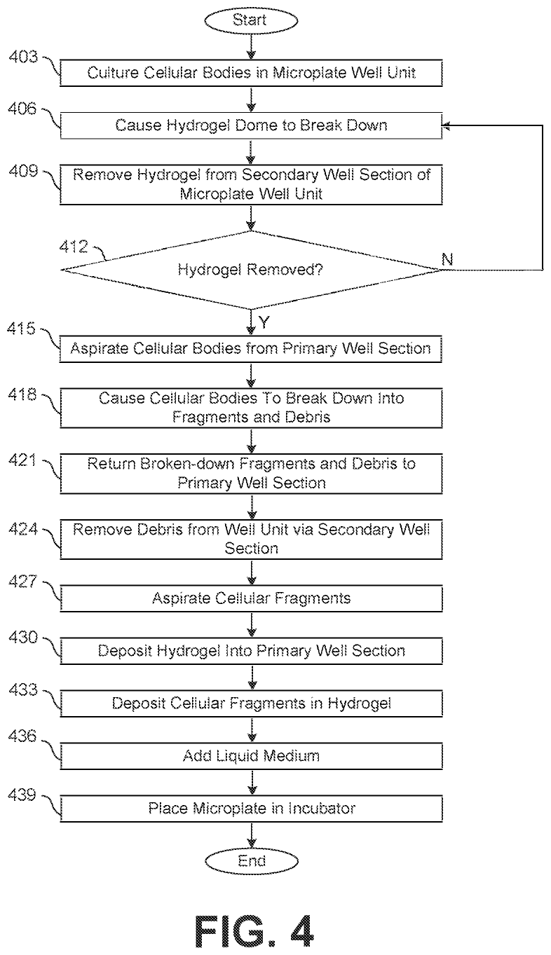

Turning now to FIG. 4, shown is a flowchart of an example method related to cell passaging with respect to a well unit 100 of a microplate in accordance to various embodiments of the present disclosure.

Beginning with step 403, cellular bodies 103 can be cultured in primary well section 112 of a well unit 100 of a microplate to provide for the growth and development of spheroids, tumoroids, organoids and/or other multi-cellular bodies. According to various embodiments, the cellular bodies 103 can be embedded in a hydrogel 106. Furthermore, one or more growth factors and supplements can be introduced into the well unit 100 of a microplate in the form of a liquid medium 109. Exemplary growth factors that can be suitable include angiopoietin, bone morphogenetic proteins (BMPs), ciliary neurotropic factor, colony stimulating factors, ephrins, epidermal growth factor, erythropoietin, fibroblast growth factors, glial-derived neurotrophic factor, hepatocyte growth factor, insulin, insulin-like growth factors, interleukins, leukemia inhibitory factor, keratinocyte growth factor, neuregulins, neurotrophins, platelet-derived growth factor, transforming growth factors, tumor necrosis factor (alpha), vascular endothelial growth factor, and/or the like. As the cellular bodies 103 grow and develop over time in the well units 100 of the microplate, passaging can be required to remove debris 309 associated with dead cells and toxic by-products, facilitate continued cell growth, reduce total cell numbers, obtain a homogenous population of smaller cellular bodies (e.g., spheroids, tumoroids, organoids, etc), and maintain the overall health of the cellular bodies 103.

At step 406, the dome of hydrogel 106 is broken down to transform or otherwise dissipate into a plurality of hydrogel fragments 303 and/or liquid form. For example, the dome of hydrogel 106 can be transformed into a liquid, thereby separating ttle cellular bodies 103 from the hydrogel dome. In some examples, adjusting a temperature (e.g., at or less than about 10° C., at or less than about 4° C.) of the hydrogel 106 based on the melting properties of the hydrogel 106 can cause the hydrogel 106 to dissipate into a plurality of hydrogel fragments 303 or otherwise liquid form.

According to various embodiments, the culture liquid medium 109 that is present in the well unit 100 following the culturing of the cellular bodies 103 can be removed from the well unit 100 and a dissociation liquid medium 109 (e.g., gentle dissociation medium) can be introduced into the well unit 100. In some examples, the microplate can be placed in an incubator for a desired amount of time (e.g., up to about twenty (20) minutes). In this example, the dissociation liquid medium 109 having a temperature that is less than or equal to a desired temperature can be introduced into the well unit 100 via the primary well section 112 or the secondary well section 115 using a liquid handler 200, as shown in FIG. 3A. In some examples, a microplate can be positioned on a temperature-controlled device (e.g., cooling plate) that is designed to adjust the temperature of the microplate such that the hydrogel 106 deposited within the corresponding well units 100 begins to liquify. As the hydrogel 106 is reduced to a liquid form and/or hydrogel fragments 303, the cellular bodies 103 are separated from the hydrogel “106.

At step 406, the hydrogel fragments 303 or otherwise liquid form of the hydrogel 106 can be removed from the secondary well section 115 of the well unit 100. In particular, as the hydrogel is reduced to fragments 303 or otherwise liquid form, the hydrogel 106 can pass through the at least one channel 118 that fluidly connects the primary well section 112 from the secondary well section 115. In some examples, the microplate can be tilted to facilitate the flow of fluid from the primary well section 112 into the secondary well section 115.

For example, the microplate can be placed on a rocking or tilting apparatus. Such apparatus can have a flat surface configured to receive a microwell plate as described here that is operably connected to a motor that can “tilt” the microwell plate about an axis, thereby raising the height of one side relative to the opposing side and facilitating the flow of fluid from the primary well section 112 (e.g., culture well) of the well unit 100 to the secondary well section 115 (e.g., culture well), or vice versa. As the cellular bodies are sized with dimensions that exceeds the dimensions of the at least one channel 118, the cellular bodies 103 remain in the primary well section 112 of the well unit 100, as can be appreciated.

The hydrogel 106 (e.g., hydrogel fragments 303) and liquid medium 109 can be removed from the secondary well section 115 using a liquid handler 200, as illustrated in FIG. 3B. Although the hydrogel 106 and the liquid medium 109 could also be removed from the primary well section 112, it is preferred to remove the liquid medium 109 and the hydrogel 106 from the secondary well section 115 in order to preserve and avoid disturbing the cellular bodies 103 that are situated within the primary well section 112.

At step 412, it can be determined whether all of the hydrogel 106 has been removed from the well unit 100. If all of the hydrogel 106 (or an acceptable amount) has been removed, the process proceeds to step 415. Otherwise, the process returns to step 406. In particular, the liquid medium 109 can be reintroduced into the well unit 100 to allow the hydrogel 106 to properly break down for removal. These steps of the workflow can be repeated as necessary to allow for the removal of the hydrogel 106 from the well unit 100. In addition, the cellular bodies that remain in the primary well section 112 of the well unit 100 can be washed by introducing and removing a liquid medium 109 to the well unit 100, as can be appreciated. This washing process can be done up to about three times.

At step 415, the cellular bodies 103 that remain in the primary well section 112 of the well unit 100 are aspirated or otherwise collected in a liquid handler 200, as illustrated in FIG. 3D.

At step 418, a liquid medium 109 (e.g., dissociation or culture medium, etc) can be added to the liquid handler 200 and the cellular bodies 103 are broken down into a plurality of cellular body fragments 306 and debris 309. In various examples, the liquid medium 109 can be used to facilitate the breakdown of the cellular bodies 103. For example, shear forces can be applied to the cellular bodies 103 as a result of harsh pipetting which causes a back and forth movement of the cellular bodies 103 within the body of the liquid handler 200. In this example, the liquid handler 200 can be manipulated a number of times (e.g., 20) to allow for the cellular bodies 103 to move within the body of the liquid handler 200, thereby causing the cellular bodies 103 to break down into a plurality of cellular body fragments 306 and debris 309 (e.g., dead cells, toxic by-product, etc). In various examples, a preferred size of the cellular body fragments 306 is around 25-500μ. However, it should be noted that other size fragments can be obtained, as can be appreciated.

At step 421, the cellular body fragments 306 and debris 309 are returned to the primary well section 112 of the well unit 100, as illustrated in FIG. 3E. At step 424, the debris 309 is removed from the well unit 100. In various examples, a liquid medium 109 can be introduced to allow for the debris 309 to be washed away from the cellular body fragments 306 and travel through the at least one channel 118 into the secondary well section 115, as illustrated in FIG. 3F. In particular, the microplate can be tilted to cause the gravitational flow of fluid through the at least one channel 118 and in between the two well sections. The debris 309 that is sized with dimensions that are less than the dimensions of the at least one channel 118 are able to pass through the channel 118 and flow into the secondary well section 115, as can be appreciated. Similarly, the cellular body fragments 306 are sized with dimensions that are greater than the dimensions of the at least one channel 118 and are thereby prevented from passing through the at least one channel 118 and remain within the primary well section 112. In various examples, the addition of the liquid medium 109, the migration of the debris 309 into the secondary well section 115, and/or removal of the debris 309 using the liquid handler 200 can be repeated as necessary to ensure the removal of the debris 309.

At step 427, the cellular body fragments 306 that remain in the primary well section 112 can be aspirated or otherwise collected using a liquid handler 200, as illustrated in FIG. 3G. In various examples, a liquid medium 109 is added to the well unit 100 to allow for safe handling and collection of the cellular body fragments 306.

In step 430, a hydrogel 106 can be deposited into the primary well section 112 of the well unit 100 to create a fresh environment for culturing cells. As can be appreciated, the hydrogel 106 as described herein can comprise Matrigel® (gelatinous protein mixture secreted by Engelbreth-Holm-Swarm mouse sarcoma cells; Corning® Life Sciences). In other aspects, hydrogels or scaffold as described herein can comprise one or more extracellular matrix components, for example a collagen or fibronectin, bioinks, gelatin, alginate, Biomimesys®, cellulose-based hydrogels, basement membrane extract (BME), or other hydrogels, scaffolds, or scaffold-free solutions as can be appreciated

At step 433, the cellular body fragments 306 are deposited in the hydrogel 106. In various examples, the cellular body fragments 306 can be deposited in the hydrogel 106 by any suitable technique including bioink droplet printing, micro-contact printing, photolithography, dip pen nanolithography, and/or pipetting, among others. In some examples, a sieve or other filter device, can be used to control the size of the cellular body fragments 306 that are deposited in and/or pre-mixed with the hydrogel 106. For example, if any cellular body fragments 306 are sized greater than desired, the sieve can be used to inhibit use of the larger cellular body fragments 306 within the new and fresh environment. Although the present method discusses the cellular body fragments 306 being deposited in the hydrogel 106 after the hydrogel 106 is deposited in the well unit 100, it should be noted that, in some embodiments, the cellular body fragments 306 can be deposited or otherwise embedded in the hydrogel 106 prior to depositing the hydrogel 106 in the well unit 100. In various examples, the hydrogel 106 (with or without the cellular body fragments 306 can be deposited on a pre-warmed microplate that is heated to a desired temperature (e.g., about thirty-seven (37) degrees Celsius), and the microplate (including the cellular body fragments) can be placed in an incubator for a desired amount of time (e.g., up to about twenty (20) minutes).

At step 436, a liquid medium 109 can be introduced into the well unit 100. The liquid medium 109 can be introduced into the well unit 100 via the primary well section 112 or the secondary well section 115. For example, the liquid medium 109 can be delivered to the primary well section 112 from the secondary well section 115 by fluid flow driven through the at least one channel 118 of the well unit 100 of the microplate. Such fluid flow can further be facilitated by manually titling the microplate plate at intervals desired by the user for periods of time desired by the user, or by other methods, such as by placement of the microplate on an automated tilting or rocker apparatus.

At step 439, the microplate can be placed in a tissue culture incubator to allow for the growth and development of the cellular body fragments 306 (e.g., spheroids, tumoroids, organoids and/or other multi-cellular bodies). Thereafter, the passaging process can proceed to completion.

### Cells

Cells according to the present disclosure can include stem cells (e.g., pluripotent stem cells), support cells, adult somatic stem cells (ASCs), patient derived material (e.g., tumoroids), and/or the like.

Cells according to the present disclosure can be mammalian cells, in particular, human, rat, or mouse cells. Cells can include various immortalized cell lines or primary cell lines (e.g., HUVEC) that are typically used in research that are known to the skilled artisan. Cells according to the present disclosure can be pluripotent or multipotent stem cells (for example, without intending to be limiting, embryonic stem cells, induced pluripotent stem cells, adult somatic stem cells (ASCs), patient derived material (e.g., tumoroids), or mesenchymal stem cells). In embodiments, stem cells according to the present disclosure can be mouse or human stem cells that are commercially available to the skilled artisan through outlets such as ATCC® or other commercial companies known in the art. Stem cells according to the present disclosure can also be human, mouse, or rat (or another organism) stem cells that were reprogrammed by the user from a source of primary cells utilizing any number of reprogramming methods and/or kits according to the art.

Support cells can be those support cells known to, for example, support stem cell culture. Without intending to be limiting, such cells can include mouse embryonic fibroblasts, induced pluripotent stem cell (iPSC) lines, embryonic stem cell lines (e.g., E5, E7, etc.), adult somatic stem cells (ASCs), patient derived material (e.g., tumoroids), patient derived organoids (e.g., intestine, liver, lpsc derived organoids, etc.), spheroids, embryoid bodies, tumoroids xenografts, various organisms (e.g., drosophila, zebrafish, etc.) or others known in the art.

The culture of various cells/cellular bodies at different stages of culture can be carried out using standard media and techniques as known in the art (for example, media including Dulbecco's modified eagle medium, stem cell media, mTeSR™, lntestiCult™ Intestinal Organoid Culture Media, or variants thereof, fetal bovine serum, and leukemia inhibitory factor for mouse iPSCs). Differentiation of stem cells to somatic cells or tissue target cells of endodermal, ectodermal, and mesodermal lineage is also known and described in the art and can be employed according to methods of the present disclosure.

### Hydrogels/Scaffolds

Hydrogels and/or scaffolds can be employed in microwell plates, systems, kits, and methods as described herein to facilitate the 3D culture, growth, and assay of cellular bodies as described herein.

In an embodiment, a hydrogel or scaffold as described herein can comprise Matrigel® (gelatinous protein mixture secreted by Engelbreth-Holm-Swarm mouse sarcoma cells; Corning® Life Sciences). In other aspects, hydrogels or scaffold as described herein can comprise one or more extracellular matrix components, for example a collagen or fibronectin, bioinks, gelatin, alginate, Biomimesys®, cellulose-based hydrogels, basement membrane extract (BME), or other hydrogels, scaffolds, or scaffold-free solutions as can be appreciated.

Unless defined otherwise, all technical and scientific terms used hereinhave the same meaning as commonly understood by one of ordinary skill in the art to which this disclosure belongs. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Many aspects of the present disclosure can be better understood with reference to the following appendices, which is hereby incorporated by reference in its entirety. The components in the drawings are not necessarily to scale, emphasis instead being placed upon clearly illustrating the principles of the present disclosure.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±20%, ±15%, ±10%, ±9%, ±8%, ±7%, ±6%, or ±5% of the specified value, e.g., about 1″ refers to the range of 0.8″ to 1.2″, 0.8″ to 1.15″, 0.9″ to 1.1″, 0.91″ to 1.09″, 0.92″ to 1.08″, 0.93″ to 1.07″, 0.94″ to 1.06″, or 0.95″ to 1.05″, unless otherwise indicated or inferred. It is understood that where “about,” “approximate,” or “at or about” is used before a quantitative value, the parameter also includes the specific quantitative value itself, unless specifically stated otherwise.

Any ratios, concentrations, amounts, and other numerical data can be expressed herein in a range format. Such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. Where the stated range includes one or both of the limits, ranges excluding either or both of those included limits are also included in the disclosure, e.g., the phrase “x toy” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘x, y, z, or greater’ should be interpreted to include the specific ranges of ‘about x’, ‘about y’, and ‘about z’ as well as the ranges of ‘greater than x’, greater than y′, and ‘greater than z’. In some aspects, the term “about” can include traditional rounding according to significant figures of the numerical value. In addition, the phrase “about ‘x’ to ‘y”’ includes “about ‘x’ to about “y.”

The term “substantially” is meant to permit deviations from the descriptive term that do not negatively impact the intended purpose. All descriptive terms used herein are implicitly understood to be modified by the word “substantially,” even if the descriptive term is not explicitly modified by the word “substantially.”

Although any methods and materials similar or equivalent to those described herein can also be used in the practice or testing of the present disclosure, the various methods and materials suitable for use with the various disclosures disclosed herein are now described. Functions or constructions well-known in the art may not be described in detail for brevity and/or clarity.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

1. A method for passaging organoids, the method comprising:
culturing one or more organoids in a hydrogel that is disposed in a well unit of a microwell plate, the well unit comprising a primary well section that is fluidly connected to a secondary well section via at least one channel, the hydrogel being disposed in the primary well section of the well unit;
dissipating the hydrogel into a plurality of hydrogel fragments, thereby separating the one or more organoids from the hydrogel;
removing the plurality of hydrogel fragments from the well unit, the one or more organoids remaining in the well unit;
breaking the one or more organoids into a plurality of organoid fragments and corresponding debris; and
creating a fresh culture environment comprising the plurality of organoid fragments.

2. The method of claim 1, wherein the at least one channel is formed by at least one gap between a bottom surface of the well unit and a bottom portion of a shared sidewall of the primary well section and the secondary well section.

3. The method of claim 2, wherein a height of the at least one gap is in a range of about ten (10) microns up to about one hundred (100) microns.

4. The method of claim 1, wherein dissipating the hydrogel into a plurality of hydrogel fragments further comprises cooling the hydrogel.

5. The method of claim 4, wherein cooling the hydrogel further comprises dispensing a liquid medium into the well unit, the liquid medium being at a temperature of about 10 degrees Celsius or less.

6. The method of claim 5, wherein the temperature is about 4 degrees Celsius or less.

7. The method of claim 1, further comprising:
collecting the one or more organoids from the primary well section of the well unit via a liquid handler;
applying at least one shear force to the one or more organoids within the liquid handler, the one or more organoids being broken into the plurality of organoid fragments and the corresponding debris as a result of the at least one shear force; and
depositing the plurality of organoid fragments and the corresponding debris into the primary well section of the well unit via the liquid handler.

8. The method of claim 7, wherein applying the at least one shear force comprises manipulating the liquid handler to cause the one or more organoids to move vertically within the liquid handler.

9. The method of claim 1, wherein removing the plurality of hydrogel fragments further comprises:
inserting a liquid handler into the secondary well section of the well unit; transferring the plurality of hydrogel fragments from the primary well section into the secondary well section of the well unit via the at least one channel; and
collecting the plurality of hydrogel fragments from the secondary well section via the liquid handler.

10. The method of claim 1, further comprising flushing the at least one channel to remove at least one of a debris fragment or an organoid fragment from the at least one channel.

11. The method of claim 1, further comprising moving the corresponding debris to the secondary well section of the well unit via the at least one channel.

12. The method of claim 1, wherein creating the fresh culture environment comprising the plurality of organoid fragments further comprises:
collecting the plurality of organoid fragments via a liquid handler; and
depositing a fresh hydrogel on a bottom surface of the primary well section of one of another well unit or the well unit.

13. The method of claim 12, wherein the plurality of organoid fragments are embedded in the fresh hydrogel prior to depositing the fresh hydrogel on the bottom surface of the primary well section.

14. The method of claim 12, wherein the plurality of organoid fragments are embedded in the fresh hydrogel after the fresh hydrogel is deposited on the bottom surface of the primary well section.

15. The method of claim 1, further comprising placing the microwell plate comprising the well unit in an incubator.

16. A method, comprising:
separating one or more cellular bodies from a hydrogel that is disposed on a bottom surface of a first well unit of a first microplate plate, the first well unit comprising a culture well and a supply well that are fluidly connected to one another via at least one channel, and the hydrogel being disposed within the culture well of the first well unit;
removing the hydrogel from the first well unit via the supply well of the first well unit, the one or more cellular bodies remaining within the culture well of the first well unit;
breaking the one or more cellular bodies into a plurality of cellular body fragments and corresponding debris;
removing the corresponding debris from the supply well of the first well unit, the plurality of cellular body fragments remaining within the culture well of the first well unit; and
creating a fresh culture environment in one of the first well unit or a second well unit, the fresh culture environment comprising the plurality of cellular body fragments.

17. The method of claim 16, wherein the at least one channel is sized and shaped to prevent passage of objects sized greater than about twenty-five microns between the culture well and the supply well.

18. The method of claim 16, wherein separating the one or more cellular bodies from the hydrogel further comprises cooling the hydrogel to a temperature that causes the hydrogel to liquify.

19. The method of claim 16, further comprising:
removing the one or more cellular bodies from the culture well via a pipette; and
applying one or more shear forces to the one or more cellular bodies via the pipette, the one or more cellular bodies being broken into the plurality of cellular body fragments and the corresponding debris based at least in part on the one or more applied shear forces.

20. The method of claim 16, wherein creating the fresh culture environment in one of the first well unit or the second well unit further comprises:
depositing a fresh hydrogel in the one of the first well unit or the second well unit, the plurality of cellular body fragments being embedded in the fresh hydrogel; and
culturing the plurality of cellular body fragments being embedded in the fresh hydrogel.
